(19) United States
(12) Patent Application Publication
(10) Pub. No.: US 2008/0268072 A1
(43) Pub. Date: Oct. 30, 2008

(54) Method of Improving Anticancer Effect of Pulsatillae Radix and a Composition Prepared by the Method

(75) Inventors: Song-Bae Kim, Chungcheongnam-Do (KR); Byeong Jun An, Daejeon-si (KR); Hye Young Kim, Chungcheongnam-do (KR); Jong Seok Kim, Chungcheongnam-Do (KR); Jong Uk Kim, Chungcheongnam-Do (KR); Seong Cheol Bang, Chungcheongnam-do (KR); Jee Hyun Lee, Daejeon-si (KR)

Correspondence Address: BIRCH STEWART KOLASCH & BIRCH, PO BOX 747, FALLS CHURCH, VA, 22040-0747, US

(73) Assignee: Song-Bae Kim, Gongju-Si, Chungecheongnam-Do (KR)

(21) Appl. No.: 11/658,891

(22) PCT Filed: Jul. 29, 2005
(86) PCT No.: PCT/KR05/02474

§ 371 Date: Jun. 5, 2008

(30) Foreign Application Priority Data
Jul. 30, 2004 (KR) 10-2004-0060096

(51) Int. Cl. A61K 36/00; C12P 1/00; A61P 31/00

(52) U.S. Cl. 424/725; 435/267

(57) ABSTRACT

The present invention relates to a composition enriched with 3-0-[O-α-L-rhamnopyranosyl-(1→2)-[0-β-D-glucopyranosyl-(1-→4)]-α-L-arabinopyranosyl]hederagenin (Code No. SB 365) of which antitumor activity is very strong by enzyme-reacting and extracting Pulsatillac radix.

### TECHNICAL FIELD

The present invention relates to a method of improving anticancer effect of Pulsatillae Radix and a composition prepared by the method.

Pulsatillae radix is a dried root of Pulsatilla koreana which belongs to Rantmculaceae (K. W. Bae, A Illustrated Pictorial Book of Korean Flora). The use of the Pulsatilla radix as oriental medicine is for cleaning of blood, astringency, hemostasis, antidiarrheal, etc. An extract of the Pulsatillae radix is reported to have antibacterial effect on amebic dysentery and trichomonas. Saponin of the Pulsatillae radix is also reported to have anticancer effect.

A dried Pulsatillae radix has about 9% of saponins. The isolated saponins until now are protoanemonin, anemonin, ranunculin, hederagenin, betulinic acid and oleanolic acid derivative and glycosides thereof. Much studies of actions of the saponins of the Pulsatillae radix are not carried out. Among them, protoanemonin was reported to have mitotoxicity (Vonderbank, F., Pharmazie, 5, 210, 1950). Ranunculin was reported to have cytotoxicity on KB cell and mechanism of cytotoxicity of the ranunculin was reported by inhibition of DNA-polymerase.

### BACKGROUND ART

The present inventors already isolated a material which inhibits formation of neoblood vessel and growth of tumor cell from the Pulsatillae radix. (Korean Patent No. 315200; Y. Kim, Planta med.). From water extract of the Pulsatillae radix, a saponin having antitumor effect was isolated and is dominated as Code No. SB365. This material has a strong inhibition effect of 80% against LL/2 cancer of BDF1 mouse. (Y. Kim et. al., Comparison of the antitumor activity of SB31-Injection (Trade name) with those of some clinically used antitumor agents. Archives of Pharmacal Research. 2004. 1; Korean Patent Appln No. 2002-0043016). The inventors isolated total 16 kinds of saponins from the Pulsatillae Radix and evaluated antitumor activities of each saponin together with combined prescriptions (Korean Patent Appln No. 2002-0043016).

### DISCLOSURE

### Technical Problem

Antitumor compositions comprising extract or antitumor ingredients of Pulsatillae radix are described in various patent specifications. For example, Korean Patent No. 72982 (Patent Publication No. 1994-234) discloses a pharmaceutical composition comprising extract of Pulsatillae radix as active ingredient of antitumor activity. In addition, Korean Patent Application No. 2002-0043016 (Patent Laid-open Publication No. 2004-9172) invented by the inventors of this invention discloses that active ingredient for antitumor activity of the Pulsatillae radix is 3-0-[0-α-L-rhamnopyranosyl-(1→2)-[0-β-D-glucopyranosyl-(1→4)]-α-L-arabinopyranosyl]hederagenin (Code No. SB 365) which is Pulsatillae Saponin D) and a antitumor composition comprising SB 365 as active ingredient. SB 365 has more strong activity against NCI-H23 cell line compared with adriamicine.

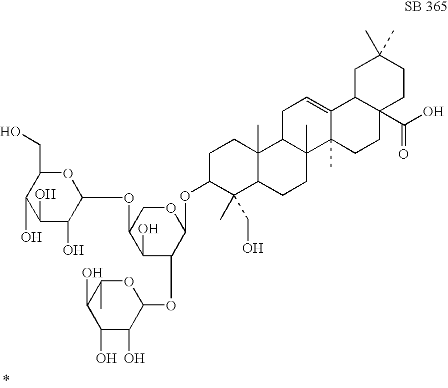

However, the content of 3-0-[0-α-L-rhamnopyranosyl-(1→2)-[0-β-D-glucopyranosyl-(1→4)]-α-L-arabinopyranosyl]hederagenin (SB 365) in Pulsatillae radix is very low and most of the Pulsatillae saponin D exists in the form of 3-0-[0-α-L-rhamnopyranosyl-(1→2)-[0-β-D-glucopyranosyl-(1→4)]-α-L-arabinopyranosyl]hederagenin 28-0-α-L-rhamnopyranosyl-(1→4-[0-β-D-glucopyranosyl-(1→6]-β-D-glucopyranosyl ester (Code No. SB 365-0 (Viqar U. A. Spectroscopic data of saponins press: CRC, 2000; Vol. 3. pp. 2520; Kang, S. S., Saponins from the root of Pulsatilla korean (Arch. Pharm. Res. 12(1), 42-47, 1989). This material is an ester in which 3 monosaccharides are bound to carboxylic group at 17-position of aglycone of SB 365. The structural formula is as follows:

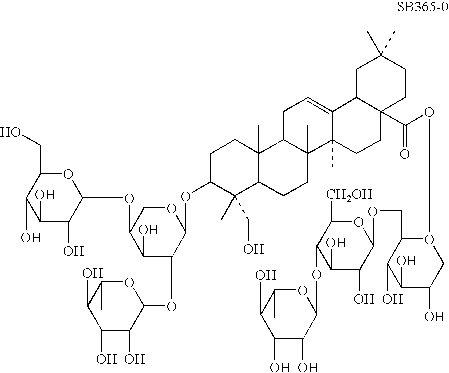

SB 365-0 has no antitumor activity. At an ordinary extraction condition of Pulsatillae radix, ester form (SB 365-0) of SB 365 is extracted as it is and has nearly no antitumor activity. In fact, a literature discloses a method in which SB 365-0 is first extracted and then is hydrolyzed into SB 365 but this method is not economical. Though an ordinary solvent extract is hydrolyzed with acid or alkali to obtain SB 365, this method is also not economical method and another ingredients of Pulsatillae radix can be chemically reacted under such acid or alkali condition. The inventors carried out a study for a long time and at last, the present invention is accomplished.

### Technical Solution

The present inventors carried out for a long time and found out the facts that Pulsatillae radix has an enzyme for disintegration of the ester linkage and in case Pulsatillae radix is enzyme-reacted (It refers to as “enzyme-reacting”.), ester of SB 365-0 is hydrolyzed to form SB 365 and after all, antitumor activity of the extract of Pulsatillae radix is drastically increased. The equation of such enzyme hydrolysis of SB 365-0 is as follows.

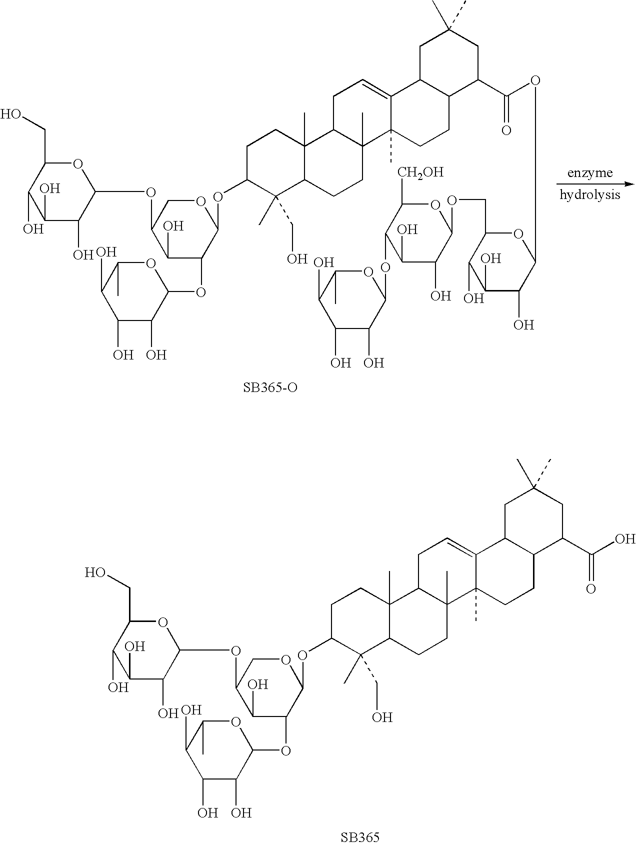

Therefore, one object of the present invention is to provide a method of increasing antitumor activity of Pulsatilla radix comprising a) enzymatically converting most SB 365-0 in Pulsatillae radix into SB 365, the antitumor ingredient; and b) extracting Pulsatillae radix of which most SB 365-0 was converted into SB 365.

Other object of the present invention is to provide a pharmaceutical composition prepared by a) enzymatically converting most SB 365-0 in Pulsatillae radix into SB 365, the antitumor ingredient, b) extracting Pulsatillae radix of which most SB 365-0 was converted into SB 365; and Pulsatillae radix extract enriched with SB 365.

Further other object of the present invention is to provide pharmaceutical composition comprising an extract of Pulsatillae radix enriched with SB 365 as main active ingredient; and an auxiliary ingredient(s) selected from the group consisting of extract of Panax ginseng, extract of Glycyrrhizae radix and extract of Pericarp of Akebia quinata.

### DESCRIPTION OF DRAWINGS

FIG. 1 shows a graph of content changes of SB365 by solvent ratio (methanol:water),

FIG. 2 shows a graph of influence of volume of water and temperature on formation of SB365,

FIG. 3 shows a graph of changes of contents of SB365 by temperature,

FIG. 4 shows a graph of changes of contents of SB365 by enzyme-reacting time, and

FIG. 5 shows a graph of tendency of formation of SB365 and reduction of SB365-0 by time.

### MODE FOR INVENTION

An optimal converting condition of SB365-0 into SB365 is to establish by volume of water, enzyme-reacting time and temperature. In addition, before establishing of optimal condition, by analysing contents of 3-0-[0-α-L-rhamnopyranosyl-(1→2)-[0-β-D-glucopyranosyl-(1→4)]-α-L-arabinopyranosyl]hederagenin (SB 365; Pulsatilla saponin D) and its substrate, 3-0-[0-α-L-rhamnopyranosyl-(1→2)-[0-β-D-glucopyranosyl-(1→4)]-α-L-arabinopyranosyl]hederagenin 28-0-α-L-rhamnopyranosyl-(1→4-[0-β-D-glucopyranosyl-(1→6]-3-D-glucopyranosyl ester (Code No. SB 365-0) (Viqar U. A., Spectroscopic data of saponins press: CRC, 2000; Vol. 3. pp. 2520; Kang S. S., Saponins from the root of Pulsatilla koreana, Arch. Pharm. Res. 12(1), 42-47, 1989), chemical relation between substrate (SB 365-0) and SB 365 is established. To obtain SB365 by using reaction medium and mechanism offered by Pulsatillae Radix itself is one of important features of the present invention.

Subjects of enzyme-reacting are Pulsatillae radix as main active ingredient (SB 31: Trade name of a pharmaceutical composition comprising extract of Pulsatillae radix, Korean Patent No. 72982, U.S. Pat. No. 6,071,521) and Ginseng radix, Glycyrrhiza glabra, Pericarp of Akebia quinata and Ulmi cortex as auxiliary ingredients.

a. Condition of Obtaining Extract Enriched with SB 365 from Pulsatillae radix

Plants which have glycosides have enzymes, that is, glycosidases which hydrolyze such glycosides. In order to maintain original structure of glycosides in extraction of glycosides, it is essential to inactivate enzymes. In an extraction condition by using an ordinary organic solvent, enzyme is inactivated and does not affect on glycosides. By contrast, in the present invention, we tried to obtain more SB 365 content than natural state by converting SB 365-0, the substrate which exists in large quantity in Pulsatillae radix and has nearly no antitumor activity through hydrolysis of SB 365-0 into SB365 by enzyme. The present invention is an improved invention of Korean Patent Application No. 10-203-0008090.

### EXPERIMENTAL EXAMPLE 1

### Enzyme-Reacting Pulsatillae Radix in Mixed Solvent of Methanol/Water

Generally, under the existence of an organic solvent, enzymatic action is difficult. Enzyme-reaction can be possible in the existence of water. To observe the effect of enzyme-reacting of Pulsatillae radix, it is important to study difference between organic solvent extraction and water extraction. Powder of Pulsatillae radix is enzyme-reacted in various concentrations of methanol in water for a certain time and then the enzyme-reacted Pulsatillae radix is extracted 3 times to obtain extract. The area percent on HPLC corresponds to SB365 is compared. The results were shown on Table 1 and FIG. 1 respectively.

As confirmed from the Table 1 and FIG. 1, extract which was enzyme-reacted and extracted in mixed solvent of methanol in water has only very low content of SB365 compared to extract which was enzyme-reacted and extracted only with water. The results show that there are no difference in SB365 content between the extract which was enzyme-reacted and extracted with pure methanol and 20% aqueous methanol and this means that enzyme hydrolyzes SB 365-0 in the Pulsatillae radix into SB365 in pure water and enzyme which hydrolyzes SB 365-0 into SB365 is very sensitive to methanol even when a small amount of methanol exists, enzyme is inactivated. In fact, 18-44 times of SB365 content in extract can be obtained in case of enzyme-reacting and extracting Pulsatillae radix with water than enzyme-reacting and extracting Pulsatillae radix in pure methanol or in aqueous methanol. Therefore, in order to obtain maximum SB365 content, Pulsatillae radix should be enzyme-reacted and extracted with pure water.

TABLE 1
HPLC peak area of SB 365 by solvent ratio

| | MeOH:H₂O | | | | | | |
|---|---|---|---|---|---|---|---|
| | 100:0 | 80:20 | 60:40 | 50:50 | 40:60 | 20:80 | 0:100 |
| first | 9054 | 8728 | 14329 | 10837 | — | 6030 | 263147 |
| second | 12664 | — | 12495 | 11365 | 11045 | 6624 | 276242 |
| third | — | 8012 | — | 8224 | 15071 | 3793 | 191076 |
| mean | 10859 | 8370 | 13412 | 10142 | 13058 | 5482 | 243488 |
| % ratio | 4.1% | 3.4% | 5.3% | 4.1% | 5.3% | 2.2% | 100% |

* % ratio: percent of HPLC peak area when the peak area in which water is used as solvent is 100.

### EXPERIMENTAL EXAMPLE 2

### Relation Between Formation of SB365 and Volume of Water

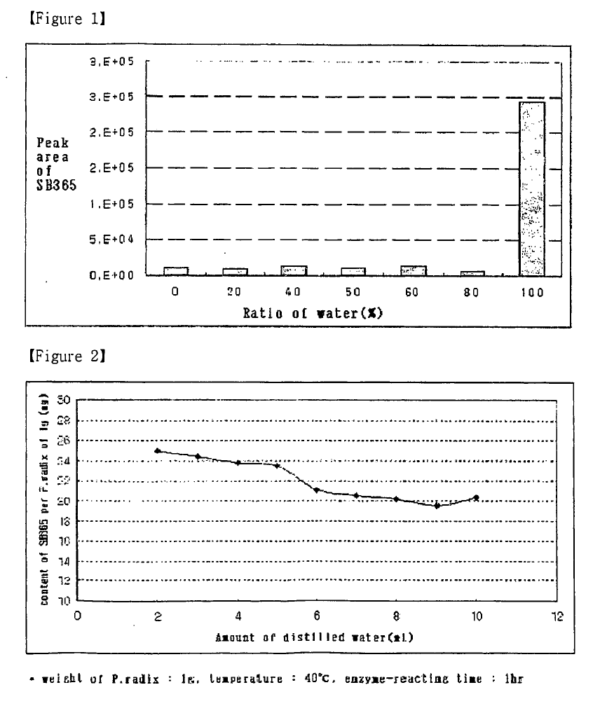

As confirmed above, existence of water in enzyme-reacting of Pulsatillae radix is a very important factor. We tried to study this factor quantitatively. First, by fixing enzyme-reaction temperature of 40° C. and time of one hour, we observed relation of forming of SB365 between Pulsatillae radix and volume of water. The results were shown in Table 2 and FIG. 2. As confirmed from the Table 2 and FIG. 2. When adding 2 times of water in weight to weight of Pulsatillae radix, formation of SB365 was excellent. The condition of 1:2 ratio in weight of Pulsatillae radix and water is concluded to be optimal condition because enzyme and concentration of SB365-0, the precursor of SB365, salines, pH and reaction condition, etc., are very close to natural state of cell of Pulsatillae radix. The ratio of 2 ml of water to 1 g of Pulsatilla radix is the minimum volume of water to be mixed and treated. 2^{˜}20 times of volume of water to 1 g of Pulsatillae radix, preferably 2^{˜}10 times of volume is added to the Pulsatillae radix and the mixture is enzyme-reacted with stirring. In order to prevent distillation of water, enzyme-reaction is preferably carried out in a chamber of 70^{˜}90% of relative humidity.

As confirmed from the Table 2 and FIG. 2, formation of SB365 tends to decrease in accordance with increasing of water content. In the case of 2-5 ml of water per 1 g of Pusatillae radix, much content o 24.9^{˜}23.4 mg/g of SB365 are formed and from 6 ml of water, the formed content of SB365 tends to decrease. This trend seemed that activity of enzyme decreases probably due to dilution of reaction medium of the reaction mixture. Substantially no change of formation of SB365 is seems to the extent of 10 times of volume of water in fermentation of Pulsatillae radix. Formation of SB365 is drastically decreased in the case of 20 times of volume of water, which data is not appeared in Table 2 and FIG. 2. Content of SB365 per 1 g of Pulsatillae radix is much more higher than in a lkown method (Korean Patent No. 72982 and U.S. Pat. No. 6,071,521).

TABLE 2
Content change of SB365 depending on content of water

| | | | |
|---|---|---|---|
| | | | Content of SB365/ |
| Volume of water | RT (min.) | Peak area (A_{T}) | 1 g of P. radix |

| | | | |
|---|---|---|---|
| 2 ml | 30.587 | 4008222 | 24.97 mg |
| 3 ml | 29.453 | 3924352 | 24.45 mg |
| 4 ml | 30.260 | 3821539 | 23.81 mg |
| 5 ml | 29.227 | 3768682 | 23.48 mg |
| 6 ml | 29.147 | 3383035 | 21.08 mg |
| 7 ml | 30.680 | 3299998 | 20.56 mg |
| 8 ml | 30.653 | 3247441 | 20.23 mg |
| 9 ml | 30.573 | 3131746 | 19.51 mg |
| 10 ml | 29.480 | 3270866 | 20.38 mg |

*As (Peak area of standard solution of SB365): 481425

### EXPERIMENTAL EXAMPLE 3

### Relation Between Enzyme-Reaction Temperature and Formation of SB365

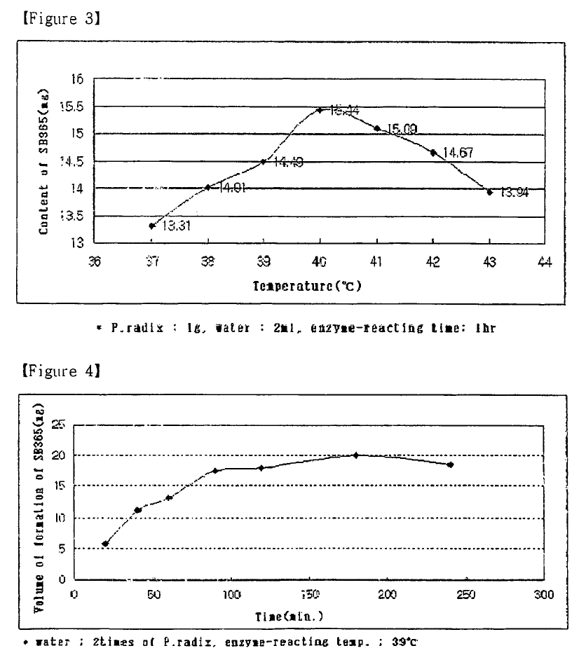

In this experiment, mixture of various raw materials of Pulsatillae radix was used. In the experiment example 2, optimal volume of water was fixed and range of reaction temperature from preliminary temperature scale is fixed. By dividing 1° C. unit of enzyme-reaction temperature, enzyme-reaction was carried out and content of SB365 in the enzyme-reaction medium was measured. The results were shown in Table 3 and FIG. 3. From Table 3 and FIG. 3, as formation of SB365 was rapidly increased from 40° C., the experiment was started from 37° C. Content of SB365 was gradually increased in accordance with rising of temperature and maximum content of 15.44 mg/g was accomplished at 40° C. In case temperature exceeded 40° C., formation of SB365 was gradually decreased. However, much content of 14.6 mg/g of SB365 was formed even at 42° C., Therefore, actually applicable temperature range in the present invention is 38^{˜}42° C.

TABLE 3
Content change of SB365 depending on temperature

| Enzyme-reaction | RT | | Content of | Mean content |
|---|---|---|---|---|
| temperature (° C.) | (min.) | Peak area (A_{T}) | SB365 | of SB365 (mg) |
| 37 | 26.72 | 3648.042 | 13.36 | 13.31 |
| | 26.71 | 3561.759 | 13.04 | |
| | 26.91 | 3695.118 | 13.53 | |
| 38 | 25.91 | 3865.115 | 14.15 | 14.01 |
| | 26.63 | 3772.591 | 13.81 | |
| | 27.27 | 3846.004 | 14.08 | |
| 39 | 26.51 | 3895.752 | 14.27 | 14.49 |
| | 26.36 | 4016.354 | 14.71 | |
| | 26.58 | 3962.139 | 14.51 | |
| 40 | 25.62 | 4236.309 | 15.51 | 15.44 |
| | 25.81 | 4183.889 | 15.32 | |
| | 25.61 | 4232.389 | 15.51 | |
| 41 | 26.34 | 4093.263 | 14.99 | 15.09 |
| | 26.18 | 4163.346 | 15.25 | |
| | 30.59 | 4106.118 | 15.04 | |
| 42 | 29.49 | 3912.663 | 14.33 | 14.67 |
| | 29.07 | 3963.894 | 14.52 | |
| | 29.35 | 4144.249 | 15.18 | |
| 43 | 29.13 | 3805.341 | 14.00 | 13.94 |
| | 28.89 | 3782.285 | 13.85 | |
| | 28.39 | 3815.679 | 13.97 | |

*As (Peak area of standard solution of SB365): 272.98

### EXPERIMENTAL EXAMPLE 4

### Changes of Formation of SB365 Depending on Enzyme-Reaction Time

The same of raw material was used as used in experimental example 3. As volume of water and temperature of enzyme-reaction were fixed, in order to obtain maximum content of SB 365, we tried to find optimal enzyme-reaction time. Result was shown in Table 4 and FIG. 4. As seen from the Table 4 and FIG. 4, Formation of SB365 was 9.1 mg in 20 min, 16.3 mg in 90 min, 20.1 mg in 180 min and 19.2 mg in 240 min, respectively. Therefore, time of enzyme-reaction is 20 min-12 hours, preferably 90 min-240 min, most preferably about 180 min. In conclusion, optimal enzyme-reaction time is 180 min. Actually range of applicable optimal enzyme-reaction time is 90-240 min. Thereafter content of SB365 tends to decrease and this means that thereafter SB365 is destroyed or enzyme is inactivated.

TABLE 4
Change of content of SB365 depending on enzyme-reaction time

| Enzyme-reaction | RT | | Content of |
|---|---|---|---|
| time (min.) | (min.) | Peak area (AT) | SB365 (mg) |
| 0 | 25.613 | 370.795 | 0.697 |
| 20 | 26.877 | 4997.575 | 9.153 |
| 40 | 27.000 | 6365.020 | 11.658 |
| 60 | 28.463 | 7370.521 | 13.499 |
| 90 | 27.003 | 8905.543 | 16.311 |
| 120 | 28.872 | 9977.757 | 18.275 |
| 180 | 28.852 | 70979.240 | 20.109 |
| 240 | 28.165 | 10489.101 | 19.211 |

*As (Peak area of standard solution of SB365): 272.98

Results of the Experiments

1. Conclusion of Enzyme-Reaction of Pulsatillae Radix Based on the Above Experiments

In enzyme-treating of a certain amount of powder of Pulsatillae radix, 2 times of volume of water was most desirable. But, in the case of increasing of 4-10 times of volume of water, considerably much amount of SB365 can be formed. Though optimal temperature is 40° C., economically meaningful amount of SB365 was formed between the temperature range of 38^{˜}42° C. when 2 times of volume of water was used and Pulsatillae radix was enzymed-reacted at 30° C. for 3 hours, yield of SB365 was 20.1 mg/g^{˜}24.5 mg/g as obtained in the experimental example. This yield corresponded to 3^{˜}5 times compared with the yield in Korean Patent No. 722982 and U.S. Pat. No. 6,071,521 (Title: A novel pharmaceutical composition having anticancer activity). The extract of Pulsatillae radix which was enzyme-reacted and extracted in the present invention is referred to as “Pu-ex”.

2. Chemistry of Formation of SB365

As volume of formation of SB365 is in proportion to temperaton and time of enzyme-reaction and is in inverse proportion to volume of water, the formation of SB 365 is definitely chemical reaction in which hydrolytic enzyme is concerned. The present inventors purely isolated SB365-0 which is seemed to a precursor of SB365 in order to study this enzyme-reaction definitely. According to the experimental example 1, SB 365 was well isolated under the solvent condition of acetonitrile:water=36:64 and showed retention time (RT) of 29-30 min. In addition, Among HPLC condition in the experimental example 1, as solvent condition of was acetonitrile:water=25:75, peak of SB365-0 was well isolated and RT was 15-16 min.

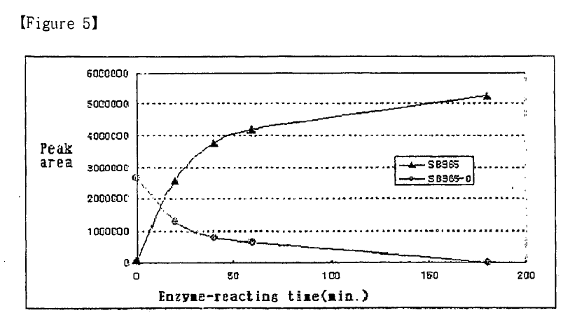

After RTs of SB365 and SB365-0 were fixed, 2 g of water was added to 1 g of Pulsatillae radix and formation of SB365 and decrease of SB365-0 were observed at 39° C. as time went on. The results were shown in Table 5 and FIG. 5. As seen from FIG. 5, formation of SB365 and decrease of SB365-0 are in inverse proportion and from the results, SB365-0 is definitely substrate of SB365. As seen that initial rate of reaction velocity is fast and thereafter reaction velocity is steady, as time goes on. The reaction is deemed to be a typical enzyme reaction.

3. From the Above Experiment Results, Enzyme-Reaction is Possible at 2-20 Times of Volume of Water to 1 g of Pulsatillae Radix, at 37˜43° C. and for 20 min˜360 min.

TABLE 5
Changes of peak area of SB365 and SB365-0 depending on time

| | |
|---|---|
| SB365 | SB365-0 |

| Time | | Peak | | Peak |
|---|---|---|---|---|
| (min.) | Rt (min.) | area (At₁) | Rt (min.) | area (At₂) |
| 0 | 28.187 | 93306 | 15.613 | 2685762 |
| 20 | 29.613 | 2615161 | 15.107 | 1297520 |
| 40 | 30.387 | 3751003 | 16.373 | 798733 |
| 60 | 30.253 | 4194653 | 16.013 | 640449 |
| 180 | 30.627 | 5254626 | 15.267 | 8499 |

* Solvent condition at HPLC analysis: SB365 (acetonitrile:water = 36:64), SB365-0 (acetonitrile:water = 25:75)

b. Solvent Extract of SB365 from Eme-Reacted Pulsatillae radix

To a paste of enzyme-reacted Pulsatillae radix, methanol is added to prepare 30^{˜}90% methanol solution. The mixture is stirred and centrifuged to obtain solution. Residue is more extracted 2 times with 30^{˜}90% methanol solution. The combined 30-90% methanol extract is dried and to this 20^{˜}50 times of voltune of pure methanol of 99% or more to 1 g of original Pulsatillae radix. The mixture is stirred and stood by. Precipitated carbohydrates and polymers are filtered out and methanol solution is distilled to obtain methanol residue. Yield of methanol residue (methanol extract) is mean 580 mg to 1 g of Pulsatillae radix. The methanol extract is used as antitumor agent itself or as combination with auxiliary ingredient(s).

In case ethanol, isopropanol or butanol is used in the same percent, the obtained extract is 478 mg, 501 mg and 411 mg, respectively. The most yield was obtained with methanol of high polarity.

In addition to Pu-ex, the main ingredient for antitumor agent, as auxiliary ingredient(s), one or more ingredients selected from the group consisting of extract of Panax ginseng, extract of Glycyrrhiza glabra, extract of Pericarp of Akebia quinata and extract of Ulmi cortex can be incorporated to obtain increased antitumor activity.

Panax ginseng has anti-stress activity, anti-diabetic activity, and various pharmacological activities. Though Panax ginseng is used as functional food for antitumor activity but such activity is very week and can not be used as pharmaceutical agent. Diacetylenes derivatives included in Panax ginseng were reported to have strong cytotoxic activity but no study on antitumor activity of the diacetylene derivatives was reported.

Glycyrrhiza glabra is used for the protection of kidney and liver, detoxication, analgesia and anti-inflammation. Though various studies carried out on protection of liver of Glycyrrhiza glabra, no study on antitumor activity was carried out. As Glycyrrhiza glabra has low toxicity and detoxication effect, we decided to add extract of Glycyrrhiza glabra to composition of the present invention.

Pericarp of Akebia quinata is a fruit of Akebia quinata and has effects on lumbago, intercostal pain, gastralgia, urethrolithiasis, menstrual irregularity and diarrhea. As Pericarp of Akebia quinata has saponins such as hederagenin and oleanolic acid. There is a possibility that Pericarp of Akebia quinata exerts synergistic effect on antitumor acivity of Pulsatillae radix.

Ulmi cortex is bark and root of Ulmus species and is used in diuresis and edema. In recent literature, Ulmi cortex is reported to prevent systemic and local anaphylaxis [Him, H. M., Shin H. Y., Choi, I. Y., Lee E. H. and Lee E. J., Action of Ulmi radicis cortex extract on systemic and local anaphylaxis on rats. Gen. Pharmacol., 31, 483-488 (1998)]. In this literature, Ulmi cortex is reported to have no toxic effect.

The present invention is explained in more detail with following examples.

### EXAMPLE 1

Each 1 g of Pulsatillae radix powder is precisely weighed and put in 9 centrifuging tubes respectively. A certain amount of water from 2 ml to 10 ml by 1 ml unit is added to each centrifuging tube and is pasted respectively. Each centrifuging tube is enzyme-reacted at 40° C. for 1 hour respectively. To each tube, each 30 ml of methanol is added and the tubes are ultrasonically stirred for 10 min and centrifuged at 300 rpm for 20 min and each upper layer is collected. To each tube, each 30 ml of methanol is added, ultrasonically stirred for 10 min and centrifuged at 300 rpm for 20 min. Each combined layer is distilled to obtain each residue. To each residue, each 30 ml of methanol for HPLC is added, dissolved and filtered to obtain each solution for HPLC analysis. Each solution is analysed by HPLC to obtain each yield of SB 365.

Content of SB365 per 1 g of P. Radix(mg)=content of SB365 in standard solution(mg/ml)×(AT/AS)×30(ml)

Solvent condition of mobile phase:acetonitrile:water=36:64
The results are shown on FIG. 2.

### EXAMPLE 2

Each 1 g of Pulsatillae radix powder is precisely weighed and put in 6 centrifuging tubes. Each 2 ml of water is added to the tubes and each mixture is pasted. The tubes are enzyme-reacted in incubators at each temperature condition of 37-42° C. at 1° C. interval for 1 hour. Thereafter, To each tube, each 30 mil of methanol is added and the tubes are ultrasonically stirred for 10 min and centrifuged at 3000 rpm for 20 min and each upper layer is collected. To each tube, each 30 ml of methanol is added, ultrasonically stirred for 10 min and centrifuged at 300 rpm for 20 min. Each combined layer is distilled to obtain each residue. To each residue, each 10 ml of methanol for HPLC is added, dissolved and filtered through 0.45 μm membrane filter to obtain each solution for HPLC analysis. Each solution is analysed by HPLC to obtain each yield of SB 365.

Content of SB365 per 1 g of P. Radix(mg)=content of SB365 in standard solution(mg/ml)×(AT/AS)×10(ml)

Solvent condition of mobile phase:acetonitrile:water=35:65
The results are shown on FIG. 3.

### EXAMPLE 3

Each 1 g of Pulsatillae radix powder is precisely weighed and put in 8 centrifuging tubes. Each 2 ml of water is added to the tubes and each mixture is pasted. The tubes are enzyme-reacted in incubators in incubation room of 39° C. for each fixed time. Thereafter, To each tube, each 30 ml of methanol is added and the tubes are ultrasonically stirred for 10 min and centrifuged at 3000 rpm for 20 min and each upper layer is collected. To each tube, each 30 ml of methanol is added, ultrasonically stirred for 10 min and centrifuged at 3000 rpm for 20 min. Each combined layer is distilled to obtain each residue. To each residue, each 5 ml of methanol for HPLC is added, dissolved and filtered through 0.45 μm membrane filter to obtain each solution for HPLC analysis. Each solution is analysed by HPLC to obtain each yield of SB 365.

Content of SB365 per 1 g of P. Radix(mg)=content of SB365 in standard solution(mg/ml)×(AT/AS)×5(ml)

Solvent condition of mobile phase:acetonitrile:water=35:65
The results are shown on FIG. 4.

### EXAMPLE 4

1 g of Pulsatillae radix powder is precisely weighed and put in a centrifuging tube. 2 ml of water is added to the tube and the mixture is pasted. The tube is enzyme-reacted in incubators in incubation room of 39° C., the optimal condition for 3 hours. Thereafter, To the tube, 30 ml of methanol is added and the tube is ultrasonically stirred for 10 min and centrifuged at 3000 rpm for 20 min and upper layer is collected. To the tube, 30 ml of methanol is added, ultrasonically stirred for 10 min and centrifuged at 300 rpm for 20 min. Combined layer is distilled to obtain a residue. To the residue, each 30 ml of methanol for HPLC is added, dissolved and filtered through 0.45 μm membrane filter to obtain a solution for HPLC analysis. The solution is analysed by HPLC to obtain yield of SB 365.

Content of SB365 per 1 g of P. radix(mg)=content of SB365 in standard solution(mg/ml)×(AT/AS)×30(ml)

Solvent condition of mobile phase:acetonitrile:water=36:64
The results are shown on FIG. 4. The best result is obtained from extract extracted for 180 min and is designated as “Pu-ex”.

### EXAMPLE 5

### Enzyme-Reacted Preparation of Natural Plants Containing Pulsatillae Radix

### Preparation of GGG-Ex

### Recipe 6

3 g of powdered Pulsatillae radix, 1.5 g of powdered Ginseng radix and 0.45 g of powdered Glycyrrhizae radix are finely mixed and 10111 of distilled water is added thereto and is pasted. The paste is added to beaker of 250 ml. The beaker is covered with gauze and cover in turn and enzyme-reacted in incubator of 39° C. and 90% of relative humidity for 3 hours. To the beaker, 50 ml of methanol is added, stirred for 10 min and filtered. To residue, each 50 ml of 80% methanol is added and extracted 2 times. Combined methanol solution is evaporated under reduced pressure to obtain a residue. To the residue, 60 ml of pure methanol is added and stirred and filtered with 0.45 μm membrane filter to remove impurities. Filtrate is evaporated under reduced pressure to obtain 2.13 g of extract. The extract is dissolved in 30 ml of physiological saline, filter through 0.22 μm millidisk cartridge filter to give solution for injection. For long term storage, the extract can be lyophilized.

### EXAMPLE 6

### Preparation of Ginseng Extract

### Preparation of “G-Ex”

To 1 g of powdered Ginseng radix, 2 ml of water is added, pasted and enzyme-reacted under the same condition with Example 4. 10 ml of methanol is added thereto, stirred and extracted. Residue is extracted with each 5 ml of 80% methanol twice. Combined methanol extract is distilled to obtain 431 mg of G-ex.

### EXAMPLE 7

### Preparation of Extract of Glycyrrhizae Radix

### Preparation of “Gg-ex”

1 g of powdered Glycyrrhizae radix is treated under the same condition with Example 6 to obtain 470 mg of Gg-ex.

### EXAMPLE 8

### Preparation of Extract of Pericarp of Akebia quinata

### Preparation of “Q-ex”

1 g of powdered Pericarp of Akebia quinata is treated under the same condition with Example 6 to obtain 553 mg of Q-ex.

### EXAMPLE 9

### Preparation of Extract of Ulmi Cortex

### Preparation of “U-ex”

1 g of Ulmi cortex is treated tinder the same condition with Example 6 to obtain 367 mg of U-ex.

Extract of Pulsatillae radix of which antitumor activity is increased of the present invention can be prepared alone or together with auxiliary ingredient(s) selected from the group consisting of Panax ginsing, Glycyrrhizae radix, Pericarp of akebia quinata and Ulmi cortex and with pharmaceutically acceptable excipient(s) into injection, tablet, powder, solution, syrup or soft capsule.

### PREPARATIVE EXAMPLE

### Recipe

### Preparative Example 1

### Recipe 1

250 ng of Pu-ex obtained from example 4 is dissolved in 50 ml of physiological saline, filtered with 0.22 μn mullkdisk cartridge filter and is filled in 5 ml of ample. 0.2 ml of this solution is injected into mouse.

### Preparative Example 2

### Recipe 2

The composition prepared by known literature of Korean Patent No. 72982 and U.S. Pat. No. 6,071,521. (SB 31: Tradename:)

### Preparative Example 3

### Recipe 3

250 mg of Pu-ex, 90 mg of G-ex and 30 mg of Gg-ex are dissolved in 50 ml of physiological saline, filtered with 0.22 μn mullkdisk cartridge filter and is used.

### Preparative Example 4

### Recipe 4

250 mg of Pu-ex, 120 mg of Q-ex and 30 mg of Gg-ex are dissolved in 50 ml of physiological saline, filtered with 0.22 μn mullkdisk cartridge filter and is used.

### Preparative Example 5

### Recipe 5

230 mg of Pu-ex, 80 mg of U-ex and 30 mg of Gg-ex are dissolved in 50 ml of physiological saline, filtered with 0.22 μn mullkdisk cartridge filter and is used.

### Preparative Example 6

### Recipe 6

The composition obtained from example 5.

### EXPERIMENTAL EXAMPLE 5

Based on Pu-ex, to extracts of raw plants obtained above, extracts of other plants are added to obtain antitumor recipes of Table 6 below. Extract ratio and administered contents of the antitumor recipes of Table 6 are followed the recipe of Korean Patent No. 72982 and U.S. Pat. No. 6,071,521. (SB 31 (Tradename) weight ratio of Pulsatillae radix:Ginseng radix:Glycyrrhizae radix=3:1.5:0.45).

Each yields of extracts and contents of extracts in each 5 ml of injections are shown in Table 6.

TABLE 6
each yields of extracts per 1 g of plants and extract contents
of each vial (5 ml)

| kinds of plants | extract | yield | contents per vial |
|---|---|---|---|
| Pulsatillae radix | Pu-ex | 580 mg/g | 25 mg/vial |
| Ginseng radix | G-ex | 431 mg/g | 9 mg/vial |
| Glycyrrhizae radix | Gg-ex | 470 mg/g | 3 mg/vial |
| Pericarp of akebia quinata | Q-ex | 553 mg/g | 12 mg/vial |
| Ulmi cortex | U-ex | 367 mg/g | 8 mg/vial |

* Ground of calculation: 20 mg of SB365/1 g of enzyme-reacted P. radix, 0.87 mg of SB365/43 mg of Pu-ex.

Contents of each extracts in 5 mil of vials in recipe 1-6 are shown in Table 7.

TABLE 7
Each recipe and antitumor activities

| | | antitumor |
|---|---|---|
| | | activity |
| recipe | contents of extracts | [IR(%)] |
| recipe 1 | Pu-ex 25 mg/5 ml | 67% |
| recipe 2 | SB31/5 ml | 60% |
| recipe 3 | (Pu-ex 25 mg + G-ex 9 mg + Gg-ex 3 mg)/5 ml | 70% |
| recipe 4 | (Pu-ex 25 mg + Q-ex 12 mg + Gg-ex 3 mg)/5 ml | 62% |
| recipe 5 | (Pu-ex 25 mg + U-ex 8 mg + Gg-ex 3 mg)/5 ml | 60% |
| recipe 6 | GGG-ex/300 ml | 71% |

* Antitumor activity [IR(%)] is mean value of determinations 3 times.
* In recipes 1, 2, 3, 4 and 5, each 0.2 ml of each recipe is administered into each mouse I.P.
* GGG-ex is dissolved in 300 ml of physiological saline, filtered with 0.2 ml mullkdisk cartridge filter and is amdinistered into each mouse I.P.

Results of Experiments

Recipe 1 which is composed of extract extracted enzyme-reacted Pulsatillae radix showed superior antitumor activity to recipe 2 which is known recipe of SB31®. Recipe 6 is an recipe in which the same weight ratio of raw plants with SB31® is enzyme-reacted at optimal condition and is extracted with methanol. Therefore, recipe 6 is quite different from the recipe of SB31® which is extracted with water without enzyme-treat.

Accordingly, recipe 6 showed excellent antitumor activity than SB31®. Recipe 4 and 5 are mixed recipes in which Q-ex or U-ex is used instead G-ex in recipe 3. Recipes 4 and 5 are not better in antitumour activity than original recipe 2 or applied recipe 6.

Conclusively, GGG-ex obtained from enzyme-reacted recipe of SB31 showed superior antitumor activity to original recipe SB31®. Therefore, Trials of stage 1 and 2 are needed.

Toxicity

Moreover, composition obtained by enzyme-reacting and methanol-extracting of the Pulsatillae radix has low toxicity than extract obtained by known water-extracting the Pulsatillae radix without enzyme-reaction of the P. radix. extract obtained by enzyme-reacting and water-extracting of the P. radix has superior antitumor activity.

Content of SB 365 in Recipe.

Recipe of Pu-ex has SB 365 in the range of 0.80-1.35 mg.

Administration Range

Based on the result of first trial for SB31®, above recipes are dissolved in 5 ml of physiological saline and is instillated in vein. Optimal instillation content is 12.15 mg/kg-29.1 mg/kg and the most optimal content is 21.87 mg/kg.

### EXPERIMENTAL EXAMPLE 7

### General Quantitative Analysis of SB365

Test sample: Accurately weighed 1 g of powdered Pulsatillae radix is added to centrifugal tube and pasted with a certain content of water. The tube was enzyme-reacted on a predetermined temperature for a predetermined time in incubator. After incubation, and 30 ml of methanol was added thereto and the mixture was stirred ultrasonically, centrifuged at 3000 rpm for 20 minutes and collected methanol solution. To residue was added 30 ml of 80% aquatic methanol. The mixture was stirred ultrasonically for 10 minutes, centrifuged at 3000 rpm for 20 minutes and collected aquatic methanol solution. Combined methanol solution was dried and distilled to obtain extract. To absolutely dried extract there added methanol for HPLC and dissolved. The methanol solution was filtered through 0.45 μm membrane filter. The filtrate was filled a vial to obtain test sample.

Standard solution: 5 mg of quantitative standard SB 365 was weighed accurately, dissolved in methanol to obtain 50 ml of standard solution.

Operation: Test sample and standard solution were tested with HPLC procedure to obtain peak area of SB365, AT and AS.

Contents of SB365 (mg)/1 g of Pulsatillae radix=content of SB365 in standard solution×(AT/AS)×volume of last used solution (ml)

Operation Conditions:

Detector: Ultraviolet wave adsorption spectrophotometer (wave: 210 nm)

Column: Watchers ODS-BP

Solvent: Mixture of acetonitrile and water (35:65 or 36:64)

Velocity: 1 ml/min

Volume: 20 μl.

### EXPERIMENTAL EXAMPLE 8

Determination of Antitumor Activity:

1. Test Animal:

Mice used in the test were BDF-1 mice of 20-23 g of body weight aged 4 weeks and were supplied form Korea Test Animal Center.

2. Breeding of Animals:

For breeding time, 22±2° C. and 65±5% of relative humidity were maintained and Light and darkness were changed as 12 hours-cycle. Water and food were supplied freely. Food without antibiotics for mouse was used.

3. Test Method

Following Teruhiro method, each LL/2 (Lewis lung carcinoma cell) 1×106/mouse was transplanted to each axilla of left forefoot of BDF-1 mouse. After 24 hours of transplant, each group consisting of 5 animals was divided. As inspecting each change of body weight of each mouse, each test sample was injected intraperitoneally for 2 weeks. After tumor volume of control group reached about 2 cm3, each volume of tumor of test group was measured and antitumor activity was measured by the following equation.

$$\begin{matrix}Tumor volume ( mm 3 ) = length ( mm ) \times area 2 ( mm 2 ) / 2 \\ Percent of inhibition of growth of tumor ( \% ) = \\ \frac{\begin{matrix}mean tumor volume of control group ( C ) - \\ mean tumor volume of test group ( T )\end{matrix}}{mean tumor volume of control group ( C )} \times 100\end{matrix}$$

4. Dose

Each 250 mg of Pu-ex and other recipes 2˜6 was dissolved in each 50 ml of physiological saline, filtered through 0.22 μm millidisk cartridge filter, divided and filled into each 10 amploules. Each 0.2 ml of each solution was injected to each animal. The test results were shown in the above Table 7.

### INDUSTRIAL APPLICABILITY

Antitumor ingredient, 3-0-[0-α-L-rhamnopyranosyl-(1→2)-[0-β-D-glucopyranosyl-(1→4)]-α-L-arabinopyranosyl]hederagenin (Code No. SB 365) comprised in Pulsatillae radix is very low and most of them exists in the form of 3-0-[0-α-L-rhamnopyranosyl-(1→2)-[0-β-D-glucopyranosyl-(1→4)]-α-L-arabinopyranosyl]hederagenin 28-0-α-L-rhamnopyranosyl-(1→4-[0-β-D-glucopyranosyl-(1→6]-β-D-glucopyranosyl ester (Code No. SB 365-0) which is inactive to tumor. Pulsatillae Radix has an enzyme for disintegration of the ester linkage and by enzyme-reacting and extracting the Pulsatillae radix, antitumor activity of Pulsatillae radix extract enriched with SB 365 is drastically increased.

1. A process for increasing antitumor activity of extract of Pulsatillae radix characterized in that by enzyme-reacting a mixture of 1 weight part of Pulsatillae radix and 2^{˜}20 weight parts of water at 37^{˜}43° C. for 20^{˜}360 minutes, 3-0-[0-α-L-rhamnopyranosyl-(1→2)-[0-β-D-glucopyranosyl-(1→4)]-α-L-arabinopyranosyl]hederagenin 28-0-α-L-rhamnopyranosyl-(1→4-[0-β-D-glucopyranosyl-(1→6]-β-D-glucopyranosyl ester (Code No. SB 365-0) which exists in large quantity in Pulsatillae radix and is inactive to tumor cell is converted to 3-0-[0-α-L-rhamnopyranosyl-(1→2)-[0-β-D-glucopyranosyl-(1→4)]-α-L-arabinopyranosyl]hederagenin (SB 365) which has strong antitumor activity and is extracted with a conventional solvent.

2. An extract of Pulsatillae radix enriched with 3-0-[0-α-L-rhamnopyranosyl-(1→2)-[0-β-D-glucopyranosyl-(1→4)]-α-L-arabinopyranosyl]hederagenin (SB 365) which has strong antitumor activity characterized in that by enzyme-reacting a mixture of 1 weight part of Pulsatillae radix and 2¹⁸ 20 weight parts of water at 37^{˜}43° C. for 20^{˜}360 minutes, 3-[0-α-L-rhamnopyranosyl-(1→2)-[0-β-D-glucopyranosyl-(1→4)]-α-L-arabinopyranosyl]hederagenin 28-0-α-L-rhamnopyranosyl-(1→4-[0-β-D-glucopyranosyl-(1→6]-β-D-glucopyranosyl ester (Code No. SB 365-0) which exists in large quantity in Pulsatillae radix and is inactive to tumor cell is converted to 3-0-[0-α-L-rhamnopyranosyl-(1→2)-[0-β-D-glucopyranosyl-(1→4)]-α-L-arabinopyranosyl]hederagenin (SB 365) which has strong antitumor activity and is extracted with a conventional solvent.

3. A pharmaceutical composition comprising an extract of Pulsatillae radix enriched with SB 365 of the claim 2 as main active ingredient; and auxiliary ingredient(s) selected from the group consisting of extract of Panax ginseng, extract of Glycyrrhizae radix and Pericarp of Akebia quinata.

4. A pharmaceutical preparation comprising composition of claim 2 or 3 as active ingredient and pharmaceutically acceptable adjuvant ingredient(s).
